SEQUENCE LISTING (1 sequence)

SEQ ID NO: 1 - protein, 20 aa, Artificial Sequence
  Synthetic polypeptide
WPSGGQMTGGKRKNSILNPI

(19) United States
(12) Patent Application Publication
(10) Pub. No.: US 2020/0340984 A1
(43) Pub. Date: Oct. 29, 2020

(54) METHODS OF QUANTIFYING CFTR PROTEIN EXPRESSION

(71) Applicant: Proteostasis Therapeutics, Inc., Boston, MA (US)

(72) Inventors: Daniel Kanmert, Boston, MA (US); Adriana Villella, Winchester, MA (US)

(21) Appl. No.: 16/958,899

(22) PCT Filed: Dec. 28, 2018
(86) PCT No.: PCT/US2018/067932

§ 371 Date: Jun. 29, 2020

Related U.S. Application Data
(60) Provisional application No. 62/645,894, filed on Mar. 21, 2018.
(60) Provisional application No. 62/616,836, filed on Jan. 12, 2018.
(60) Provisional application No. 62/611,763, filed on Dec. 29, 2017.

(51) Int. Cl. G01N 33/543; C07K 7/08

(52) U.S. Cl. CPC G01N 33/543; C07K 7/08

(57) ABSTRACT

The present disclosure is directed in part to methods of detecting and quantifying cystic fibrosis transmembrane conductance regulator (CFTR) protein expression in a sample, e.g., by an Enzyme-Linked Immunosorbent Assay (ELISA) or an AlphaLISA®, a fusion polypeptide capable of binding to a capture antibody and a detection antibody, and a kit for performing an ELISA or an AlphaLISA® to detect CFTR protein.

### CROSS REFERENCE TO RELATED APPLICATIONS

This application claims the benefit of, and priority to, U.S. Provisional Application No. 62/611,763, filed Dec. 29, 2017; U.S. Provisional Application No. 62/616,836, filed Jan. 12, 2018; and U.S. Provisional Application No. 62/645,894, filed Mar. 21, 2018, the contents of each of which are hereby incorporated by reference in their entirety.

### BACKGROUND

Cystic fibrosis (CF) is an autosomal recessive, multisystem disease caused by defects in the cystic fibrosis transmembrane conductance regulator (CFTR) gene. Normal CFTR protein channels chloride and bicarbonate ions across the cell membrane of epithelial cells, thereby regulating fluid balance throughout the body, including the lungs, sinuses, pancreas, intestine, reproductive system, and sweat glands. Following the identification of the CFTR gene as the cause of CF, more than 2000 mutations have been identified and approximately 281 are known to affect the quantity or function (and sometimes both) of CFTR proteins at the epithelial cell surface. The CF disease-causing mutations can result in reduced CFTR biosynthesis, production of a misfolded or unstable protein, and translation of a gating-defective or conductance-defective protein or non-functional protein. Overall, the mutations can be classified into one or more of 6 major classes (I-VI) based on the effects of the mutations on CFTR biosynthesis or protein function. For example, the most common CF-causing mutation, deletion of a single amino acid phenylalanine at position 508 (F508del), is a Class II (defective protein and trafficking mutation), Class III (defective channel regulation or channel gating), and Class VI (increased turnover of CFTR channel at the cell surface) mutation; while glycine551 substitution to aspartic acid (G551D) is a Class III (defective channel gating) mutation.

Recent therapeutic advances have addressed some of the underlying defects in CFTR mutations. Potentiators are a class of small molecule CFTR modulators which improve gating and/or conductance-defective mutations. The potentiator ivacaftor was demonstrated to improve lung function in CF patients with the gating mutation G551D. Correctors are another class of CFTR modulators which assist in the folding and/or trafficking of F508del protein to the cell membrane surface. A beneficial improvement in lung function was observed when a combination of ivacaftor (a potentiator) and lumacaftor (a corrector) was administered to CF patients who were F508del homozygous, but not to CF patients who were F508del heterozygous. Such a treatment difference may be due to the limited amount of CFTR protein in the F508del heterozygous patients.

Another class of CFTR modulators, termed amplifiers, differ from both potentiators and correctors in its mechanism. Amplifiers affect the biosynthesis of CFTR by increasing the translation of CFTR protein and slowing the decay of CFTR mRNA. The effects of amplifiers complement current corrector therapies by providing more substrate to further improve the function of CFTR. In addition, amplifiers may enhance CFTR biosynthesis independent of CFTR mutation, suggesting that amplifiers may have a beneficial effect across multiple different CFTR mutations not addressed by current therapies.

There remains a need in the art for methods of quantifying CFTR protein expression, for example, to facilitate the clinical development of CFTR modulator compounds.

### SUMMARY

The disclosure relates, in part, to a method for quantifying CFTR protein expression in a sample, the method comprising the steps of adding a biological sample to a container comprising: (a) adding a sample containing the CFTR protein to a container comprising a capture antibody, wherein the capture antibody is UNC596; (b) allowing the CFTR protein to bind the UNC596 antibody to form a CFTR protein-UNC596 complex, (c) adding a detection antibody to the CFTR protein-UNC596 complex, wherein the detection antibody is UNC450; and (d) detecting binding of the UNC450 antibody to the CFTR protein-UNC596 complex.

In some embodiments, the method further comprises comparing the amount of the binding of the UNC450 antibody to the CFTR protein-UNC596 complex to a standard curve generated using: (a) a polypeptide comprising a region exhibiting at least 90% sequence identity to WPSGGQMTGGKRKNSILNPI (SEQ ID NO:1) or to a portion thereof, wherein the portion comprises 15-19 amino acids; (b) a polypeptide exhibiting at least 90% sequence identity to WPSGGQMTGGKRKNSILNPI (SEQ ID NO:1) or to a portion thereof, wherein the portion comprises 15-19 amino acids; or (c) a polypeptide comprising a first region having at least 90% sequence identity to amino acids 1-8 of SEQ ID NO:1 and a second region having at least 90% sequence identity to amino acids 11-20 of SEQ ID NO:1, wherein the polypeptide comprises fewer than 1000 amino acids.

Further, the disclosure relates in part to a polypeptide comprising a first region having at least 90% sequence identity to amino acids 1-8 of SEQ ID NO:1 and a second region having at least 90% sequence identity to amino acids 11-20 of SEQ ID NO:1, wherein the polypeptide comprises fewer than 1000 amino acids. For example, disclosed herein is a polypeptide exhibiting at least 90% sequence identity to WPSGGQMTGGKRKNSILNPI (SEQ ID NO:1) or to a portion thereof, wherein the portion comprises 15-19 amino acids.

Also disclosed herein is a method for generating a standard curve for an Enzyme-Linked Immunosorbent Assay (ELISA) or an AlphaLISA® for detecting a cystic fibrosis transmembrane conductance regulator (CFTR), the method comprising (a) adding a polypeptide disclosed herein to a capture antibody (e.g., UNC596); (b) allowing the polypeptide to bind the capture antibody to form a polypeptide-capture antibody complex, (c) adding a detection antibody (e.g., UNC450) to the polypeptide-capture antibody complex; and (d) detecting binding of the detection antibody to the polypeptide-capture antibody complex. In some embodiments, the method further comprises the steps of (e) repeating steps (a) through (d) using varying concentrations of the polypeptide; and (f) generating a standard curve based upon the binding of the polypeptide at the varying concentrations.

Also provided herein is a kit for performing an ELISA or an AlphaLISA® to detect CFTR protein, the kit comprising: (a) a polypeptide disclosed herein; (b) a UNC596 antibody; and (c) a UNC450 antibody.

### BRIEF DESCRIPTION OF THE DRAWINGS

FIG. 1A depicts the domains of cystic fibrosis transmembrane conductance regulator (CFTR) protein. FIG. 1B shows CFTR ELISA results performed on cystic fibrosis bronchial epithelial (CFBE) parental, CFBE WT, and CFBE F508del cells using the UNC596/UNC154 antibody combination, with buffer control. FIG. 1C shows CFTR ELISA results performed on CFBE parental, CFBE WT, and CFBE F508del cells using the UNC596/UNC217 antibody combination, with buffer control. FIG. 1D shows CFTR ELISA results performed on CFBE parental, CFBE WT, and CFBE F508del cells using the UNC596/UNC450 antibody combination, with buffer control. FIG. 1E shows CFTR ELISA results performed on HBE wild type and HBE G542X/G542X cell lysates, with buffer control.

FIG. 2A shows CFTR/Actin RNA fold changes in wild type and F508del human bronchial epithelial cells (HBEs) treated with 1, 3, and 10 μM of an amplifier compound (PTI-CH) or DMSO (vehicle) for 24 hours. FIG. 2B shows CFTR protein levels in wild type and F508del human bronchial epithelial cells (HBEs) treated with 1, 3, and 10 μM of amplifier or DMSO (vehicle) for 24 hours, as assayed by CFTR ELISA. FIG. 2C shows Western blot analysis for CFTR (with Actin control) for wild type human bronchial epithelial cells (HBEs) treated with 1, 3, or 10 μM of amplifier or DMSO (vehicle) for 24 hours. FIG. 2D shows Western blot analysis for CFTR (with Actin control) for F508del type human bronchial epithelial cells (HBEs) treated 1, 3, and 10 μM of amplifier or DMSO (vehicle) for 24 hours.

FIG. 3A shows fold changes in CFTR/Actin RNA in differentiated nasal cells obtained from healthy subjects (WT-HNEs) and those obtained from homozygous F508del donors (CF-HNEs) treated with an amplifier compound (10 μM PTI-CH) or DMSO (vehicle) for 24 hours, as assayed by qPCR. FIG. 3B shows CFTR protein expression in differentiated nasal cells obtained from healthy subjects (WT-HNEs) and those obtained from homozygous F508del donors (CF-HNEs) treated with an amplifier compound (10 μM PTI-CH) or DMSO (vehicle) for 24 hours, as assayed by CFTR ELISA.

FIG. 4A shows CFTR mRNA expression, as measured by qPCR, in nasal cells collected from healthy volunteers. FIG. 4B shows CFTR protein expression, as measured by CFTR ELISA, in nasal cells collected from healthy volunteers. FIG. 4C shows a correlation between CFTR protein (Y-Axis) and CFTR mRNA (X-Axis) in nasal cells collected from healthy volunteers.

FIG. 5A shows the fold change in CFTR expression over a range of amplifier (PTI-CH) concentrations. Fold change measurements taken using UNC596 as the biotinylated (donor) antibody (squares) are similar to fold change measurements taken using UNC450 as the biotinylated (donor) antibody (triangles). FIG. 5B shows the fold change in CFTR expression when 10 μM amplifier (PTI-CH) is used as detected by a standard ELISA assay. The measurement of fold change in CFTR protein as detected using a standard ELISA is similar to that detected using an AlphaLISA®.

FIG. 6A shows standard curves for a CFTR AlphaLISA® assay. The standard curves were similar whether UNC596 was the biotinylated (donor) antibody (circles) or UNC450 was the biotinylated (donor) antibody (squares). FIG. 6B shows standard curves for a CFTR AlphaLISA® assay. The standard curves were similar whether IP buffer or 1× AlphaLISA® assay buffer was used.

### DETAILED DESCRIPTION

As used herein, the words “a” and “an” are meant to include one or more unless otherwise specified. For example, the term “an agent” encompasses both a single agent and a combination of two or more agents.

The term “modulating” encompasses increasing, enhancing, inhibiting, decreasing, suppressing, and the like. The terms “increasing” and “enhancing” mean to cause a net gain by either direct or indirect means. As used herein, the terms “inhibiting” and “decreasing” encompass causing a net decrease by either direct or indirect means.

The present disclosure is based, at least in part, on the discovery of a CFTR detection assay (e.g., a sandwich assay such as an ELISA or AlphaLISA®) to quantitate changes in CFTR protein levels. For example, using the mouse monoclonal antibodies UNC596 (capture antibody) and alkaline-phosphatase conjugated UNC450 (detection antibody), both wild type CFTR and F508del CFTR protein in cystic fibrosis bronchial epithelial (CFBE) cells overexpressing either wild type CFTR or F508del CFTR, respectively, were detected.

A fusion peptide comprising sequences from the CFTR protein, recognized by both UNC450 and UNC596, was developed as a reagent to produce a standard curve. The fusion protein has the sequence WPSGGQMTGGKRKNSILNPI (SEQ ID NO:1). UNC596 recognizes WPSGGQMT (amino acids 1-8 of SEQ ID NO:1), and UNC450 recognizes KRKNSILNPI (amino acids 11-20 of SEQ ID NO:1). The ELISA and AlphaLISA® also detected endogenous wild type and F508del CFTR protein in primary cells from human bronchial epithelial (HBE) and nasal epithelial (HNE) origin.

Further, the ELISA and AlphaLISA® detected pharmacologically-induced changes in the HBE and HNE cultures treated with an amplifier. The utility of the ELISA was further evaluated in healthy subjects by collecting nasal epithelial cells from the inferior turbinate of the nose and measuring CFTR mRNA and protein levels. The disclosed CFTR ELISA and AlphaLISA® enables, for example, quantification of CFTR protein expression to monitor the effects of CFTR modulators during clinical development.

### 1. Polypeptide for ELISA or AlphaLISA® Standard Curve

The disclosure relates in part to a polypeptide for producing a standard curve for an ELISA or AlphaLISA® for the detection of CFTR. In some embodiments, disclosed herein is a polypeptide comprising a first region having at least 90% sequence identity to amino acids 1-8 of SEQ ID NO:1 and a second region having at least 90% sequence identity to amino acids 11-20 of SEQ ID NO:1, wherein the polypeptide comprises fewer than 1000 amino acids. In certain embodiments, the polypeptide comprises fewer than 900 amino acids, fewer than 800 amino acids, fewer than 700 amino acids, fewer than 600 amino acids, fewer than 500 amino acids, fewer than 400 amino acids, fewer than 300 amino acids, fewer than 200 amino acids, fewer than 100 amino acids, fewer than 50 amino acids, fewer than 40 amino acids, fewer than 30 amino acids, or 20 amino acids or fewer.

In some embodiments, disclosed herein is a polypeptide comprising a first region having at least 90% sequence identity to amino acids 1-8 of SEQ ID NO:1 and a second region having at least 90% sequence identity to amino acids 11-20 of SEQ ID NO:1, where an amino acid linker of between 1 and 100 amino acids is disposed between the first and second region. In certain embodiments, the linker comprises between 1 and 2 amino acids, between 1 and 5 amino acids, between 1 and 10 amino acids, between 1 and 20 amino acids, between 1 and 50 amino acids, and between 1 and 75 amino acids. In certain embodiments, the linker comprises between 2 and 5 amino acids, between 2 and 10 amino acids, between 2 and 20 amino acids, between 2 and 50 amino acids, and between 2 and 75 amino acids. In certain embodiments, the linker comprises between 5 and 10 amino acids, between 5 and 20 amino acids, between 5 and 50 amino acids, and between 5 and 75 amino acids.

For example, in some embodiments, disclosed herein is a polypeptide comprising a region exhibiting at least 90% sequence identity to WPSGGQMTGGKRKNSILNPI (SEQ ID NO:1) or to a portion of the region, wherein the portion comprises 15-19 amino acids (e.g., 15 amino acids, 16 amino acids, 17 amino acids, 18 amino acids, or 19 amino acids). In some embodiments, disclosed herein is a polypeptide exhibiting at least 90% sequence identity to WPSGGQMTGGKRKNSILNPI (SEQ ID NO:1) or to a portion thereof, wherein the portion comprises 15-19 amino acids (e.g., 15 amino acids, 16 amino acids, 17 amino acids, 18 amino acids, or 19 amino acids).

In some embodiments, the at least 90% sequence identity is at least 95% sequence identity. In some embodiments, the at least 90% sequence identity is at least 98% sequence identity. In some embodiments, the at least 90% sequence identity is at least 99% sequence identity. In some embodiments, a polypeptide disclosed herein is capable of binding to a UNC596 antibody and a UNC450 antibody.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=-3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

### 2. Methods of Making a Polypeptide

Methods for producing polypeptides, for example, those disclosed herein, are known in the art. In certain embodiments, the polypeptides are chemically synthesized using techniques such as liquid-phase or solid-phase peptide synthesis.

In other embodiments, DNA molecules encoding the polypeptide can be synthesized chemically or by recombinant DNA methodologies. For example, the DNA sequence encoding the polypeptide can be cloned using polymerase chain reaction (PCR) techniques, using the appropriate synthetic nucleic acid primers. The resulting DNA molecules can be ligated to other appropriate nucleotide sequences, including, for example, expression control sequences, to produce conventional gene expression constructs (i.e., expression vectors) encoding the desired polypeptide. Production of defined gene constructs is within routine skill in the art.

Nucleic acids encoding desired polypeptides can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the polypeptides.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A polypeptide can be produced by growing (culturing) a host cell transfected with an expression vector encoding the polypeptide, under conditions that permit its expression. Following expression, the polypeptide can be harvested and purified or isolated using techniques known in the art, e.g., affinity tags such as glutathione-S-transferase (GST) or histidine tags.

### 3. Detection of CFTR by ELISA or AlphaLISA®

According to the methods of the invention, the concentration of CFTR protein may be quantitated in a bodily fluid sample using a capture antibody and a detection antibody. The methods and compositions of the present invention can be used to detect the concentration of CFTR in a sample, for example a clinical sample such as primary bronchial or nasal epithelial cells, for the evaluation of treatment efficacy. For example, the methods and compositions described herein can be used as a biomarker to evaluate the efficacy of an amplifier treatment. In some embodiments, the methods and compositions described herein can be used to predict who might respond to a certain therapy.

According to the methods of the invention, the test sample used in the detection of CFTR can be a cultured cell or a body fluid or tissue sample (e.g., a swab or a biopsy), including, but not limited to, primary bronchial epithelial cells, nasal epithelial cells, cells from the digestive or reproductive organs (e.g., rectum), and skin. Methods of obtaining a body fluid or tissue sample from a subject are known to those skilled in the art.

According to the methods of the invention, CFTR is detected and quantified using a “sandwich” assay, such as ELISA. In this embodiment, two antibodies that specifically bind to non-overlapping sites (“epitopes”) on CFTR are used. Typically, a capture antibody is immobilized on a solid surface where it binds with and captures CFTR. A second antibody is detectably labeled, for example, with a fluorophore, enzyme, or colored particle, such that binding of the second antibody to the CFTR-complex indicates that CFTR has been captured. The intensity of the signal is proportional to the concentration of CFTR in the sample. The second antibody is therefore also referred to herein as the detection antibody.

Such assay procedures can be referred to as two-site immunometric assay methods, i.e., “sandwich immunoassays.” As is known in the art, the capture and detection antibodies can be contacted with the test sample simultaneously or sequentially. Sequential methods, sometimes referred to as the “forward” method, can be accomplished by incubating the capture antibody with the sample, and adding the labeled detection antibody at a predetermined time thereafter. Alternatively, the labeled detection antibody can be incubated with the sample first and then the sample can be exposed to the capture antibody (sometimes referred to as the “reverse” method). After any necessary incubation(s), which may be of short duration, the label is detected and may also be measured. Such assays may be implemented in many specific formats known to those of skill in the art, including through use of various high throughput clinical laboratory analyzers or with point of care or home testing devices.

In one embodiment, a lateral flow device may be used in the sandwich format, wherein the presence of CFTR above a baseline sensitivity level in a biological sample will permit formation of a sandwich interaction upstream of or at the capture zone in the lateral flow assay. See, for example, U.S. Pat. No. 6,485,982. The capture zone as used herein may contain capture antibody molecules, suitable for capturing CFTR, or immobilized avidin or the like for capture of a biotinylated complex. See, for example, U.S. Pat. No. 6,319,676. The device may also incorporate a luminescent label suitable for capture in the capture zone, the concentration of CFTR being proportional to the intensity of the signal at the capture site. Suitable labels include fluorescent labels immobilized on polystyrene microspheres. Colored particles also may be used.

Other assay formats that may be used in the methods of the invention include, but are not limited to, flow-through devices. See, for example, U.S. Pat. No. 4,632,901. In a flow-through assay, an antibody is immobilized to a defined area on a membrane surface. This membrane is then overlaid on an absorbent layer that acts as a reservoir to pump sample volume through the device. Following immobilization, the remaining protein-binding sites on the membrane are blocked to minimize non-specific interactions. In operation, a biological sample is added to the membrane and filters through, allowing any analyte specific to the antibody in the sample to bind to the immobilized antibody. In a second step, a labeled secondary antibody may be added or released that reacts with captured marker to complete the sandwich. Alternatively, the secondary antibody can be mixed with the sample and added in a single step. If CFTR is present, a colored spot develops on the surface of the membrane.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984), J. Clin. Chem. Clin. Biochem. 22:895-904.

In a “sandwich ELISA,” an antibody (e.g., anti-CFTR) is linked to a solid phase (i.e., a microtiter plate) and exposed to a biological sample containing antigen (e.g., CFTR). The solid phase is then washed to remove unbound antigen. A labeled antibody (e.g., enzyme linked) is then bound to the bound antigen, forming an antibody-antigen-antibody sandwich. Examples of enzymes that can be linked to the antibody are alkaline phosphatase, horseradish peroxidase, luciferase, urease, and □-galactosidase. The enzyme-linked antibody reacts with a substrate to generate a colored reaction product that can be measured. This measurement can be used to derive the concentration of CFTR present in a sample, for example, by comparing the measurement to a CFTR standard curve.

In certain embodiments, about 1-5 μg/mL (e.g., about 1 to 3 μg/mL, about 3 to 5 μg/mL, about 2 to 4 μg/mL, about 2 to 3 μg/mL or about 3 to 4 μg/mL) capture antibody is incubated on a solid surface (e.g., a microtiter plate) for about 6 hours to about 18 hours (e.g., about 6 to about 10 hours, about 8 to about 12 hours, about 12 to about 18 hours). In certain embodiments, the incubation is performed at about 4° C. In certain embodiments, the sample is incubated with the capture antibody for about 1 to about 4 hours (e.g., about 1 to 3 hours, about 2 to 4 hours, about 2 to 3 hours), followed by about 1 to about 4 hours (e.g., about 1 to 3 hours, about 2 to 4 hours, about 2 to 3 hours) with detection antibody at 1-5 μg/mL (e.g., about 1 to 3 μg/mL, about 3 to 5 μg/mL, about 2 to 4 μg/mL, about 2 to 3 μg/mL or about 3 to 4 μg/mL). In certain embodiments, the upper (LLOQ) and lower limit of quantitation (LLOQ) are about 800 to 1200 ng/mL (e.g., about 800 to 1000 ng/mL, about 800 to 1100 ng/mL, about 900 to 1100 ng/mL, about 1000 to 1200 ng/mL, about 1000 to 1100 ng/mL, about 1000 ng/mL) and about 2 to 5 ng/mL (e.g., about 2 to 4 ng/mL, about 3 to 4 ng/mL, about 3 to 5 ng/mL, about 3.7 ng/mL) respectively. In certain embodiments, the % CV is <15% across the linear dilution range of the standard curve.

In certain embodiments, a variation on an ELISA assay known as an AlphaLISA® immunoassay can be used. (PerkinElmer, Waltham, Mass., see, doi.org/10.1038/nmeth.f.230.) In this assay, streptavidin-coated donor beads containing a photosensitizer (e.g., phthalocyanine) are added to a mixture of analyte (e.g., the CFTR protein), biotinylated antibody that recognizes an analyte, and acceptor beads conjugated to a second antibody that also recognizes the analyte. The AlphaLISA® assay uses luminescent oxygen-channeling chemistry in which laser irradiation of donor beads causes chemiluminescent emission from the acceptor bead. Thus, when the analyte is present, the proximity of the donor and acceptor beads triggers a reaction that results in chemiluminescent emission from the acceptor bead. The signal generated is proportional to the amount of analyte in the sample.

In embodiments in which phthalocyanine is used as the photosensitizer, phthalocyanine converts ambient 02 to an excited and reactive form upon illumination at 680 nm. If an acceptor bead is within proximity, energy is transferred from the oxygen resulting in light production at 615 nm.

In certain embodiments, the disclosure relates to a method for generating a standard curve for an AlphaLISA® for detecting a cystic fibrosis transmembrane conductance regulator (CFTR), the method comprising adding a polypeptide as described herein to a mixture of streptavidin-coated donor beads containing a photosensitizer (e.g., phthalocyanine), biotinylated anti-CFTR antibody, and acceptor beads conjugated to a second anti-CFTR antibody. The method may also include varying the concentration of the polypeptide; and generating a standard curve based upon the binding of the polypeptide at the varying concentrations.

In certain embodiments, the disclosure relates to a method for quantifying CFTR protein expression in a sample, the method comprising adding streptavidin-coated donor beads containing a photosensitizer (e.g., phthalocyanine) to a mixture of a sample comprising the CFTR protein, biotinylated anti-CFTR antibody, and acceptor beads conjugated to a second anti-CFTR antibody, and detecting a chemiluminescent emission signal, wherein the signal generated in proportional to the amount of CFTR protein in the sample.

In certain embodiments, UNC450 is associated with a donor bead and UNC596 is associated with an acceptor bead. In other embodiments, UNC596 is associated with a donor bead and UNC450 is associated with an acceptor bead.

a. Antibodies

In certain embodiments, monoclonal antibodies are used as capture and detection antibodies. A monoclonal antibody refers to an antibody that is derived from a single clone, including any eukaryotic, prokaryotic, or phage clone. The monoclonal antibody may comprise, or consist of, two proteins, i.e., heavy and light chains. The monoclonal antibody can be prepared using one of a wide variety of techniques known in the art including the use of hybridoma, recombinant, and phage display technologies, or a combination thereof.

Anti-CFTR monoclonal antibodies may be prepared using any known methodology, including the seminal hybridoma methods, such as those described by Kohler and Milstein (1975), Nature. 256:495. In a hybridoma method, a mouse, hamster, or other appropriate host animal is immunized with an immunizing agent to elicit lymphocytes that produce or are capable of producing antibodies that will specifically bind to the immunizing agent. Alternatively, the lymphocytes may be immunized in vitro.

The immunizing agent will typically include at least a portion of the CFTR protein or a fusion protein thereof. For example, synthetic polypeptide or recombinant polypeptide comprising amino acids 1-8 and 11-20 of SEQ ID NO:1 may be used. The immunizing agent may be administered to a mammal with or without adjuvant according to any of a variety of standard methods.

In certain embodiments, the capture antibody binds to amino acids 1-8 of SEQ ID NO: 1, and the detection antibody binds to amino acids 11-20 of SEQ ID NO: 1. In other embodiments, the capture antibody binds to amino acids 11-20 of SEQ ID NO: 1, and the detection antibody binds to amino acids 1-8 of SEQ ID NO: 1.

In certain embodiments, the capture antibody is the anti-CFTR mouse monoclonal antibody, UNC596 and the labeled detection antibody is a second anti-CFTR mouse monoclonal antibody, UNC450. In certain embodiments, the capture antibody is the anti-CFTR mouse monoclonal antibody, UNC450 and the labeled detection antibody is a second anti-CFTR mouse monoclonal antibody, UNC596. UNC450 and UNC596 are available through Cystic Fibrosis Foundation Therapeutics (CFFT) and the University of North Carolina (UNC), see, e.g., www.unc.edu/˜tjjensen/CFADP/antibodies.html. (See also, Cui et al. (2007) Journal of Molecular Biology 365(4):981-994; He et al. (2008) J. Biol Chem 283(39):26383-90; and Hegedus et al. (2009) Biochim Biophys Acta 1788:1341-1349.) Other antibodies that recognize the epitopes (amino acids 1-8 and 11-20 of SEQ ID NO:1) as described above also may be used.

b. Capture

The important property of the capture antibody is that it provides a means of separation from the remainder of the test mixture. Accordingly, as is understood in the art, the capture antibody can be introduced to the assay in an already immobilized or insoluble form, that is, a form which enables separation of the complex from the remainder of the test solution. Alternatively, immobilization may be done by capture of an immune complex comprising CFTR subsequent to introduction of a soluble form of the capture antibody to the sample. Examples of immobilized capture antibody are antibodies covalently or noncovalently attached to a solid phase such as a magnetic particle, a latex particle, a microtiter plate well, a membrane, a chip, a bead, a cuvette, an array, or other reaction vessel or holder. Examples of a soluble capture antibody is an antibody which has been chemically modified with a ligand, e.g., a hapten, biotin, or the like, and which acts as a hook to permit selective capture of complex including CFTR. Methods of coupling the capture antibody to a solid phase are well known in the art. These methods can employ bifunctional linking agents, for example, or the solid phase can be derivatized with a reactive group, such as an epoxide or an imidazole, that will bind the molecule on contact. Biospecific capture reagents against different target proteins can be mixed in the same place, or they can be attached to solid phases in different physical or addressable locations.

c. Labels

According to the methods of the invention, the label used can be selected from any of those known conventionally in the art. Preferred labels are those that permit more precise quantitation. Examples of labels include but are not limited to a fluorescent moiety, an enzyme, an electrochemically active species, a radioactive isotope, a chemiluminescent molecule, a latex or gold particle, a detectable ligand (e.g., detectable by secondary binding of a labeled binding partner for the ligand), etc. In a preferred embodiment, the label is an enzyme or a fluorescent molecule. Methods for affixing the label to the antibody are well known in the art, and include covalent and non-covalent linkage.

In one embodiment, a detection antibody can be labeled with a fluorescent compound. When the fluorescently labeled detection antibody is exposed to light of the proper wavelength, its presence can then be detected by the fluorescence emitted. Among the most commonly used fluorescent labeling compounds are Cy3 and Cy5 (water-soluble fluorescent dyes of the cyanine dye family-“Cy” dyes), fluorescein isothiocyanate, rhodamine, phycoerytherin, phycocyanin, allophycocyanin, o-phthalaldehyde and fluorescamine.

In another embodiment, the detection antibody is detectably labeled by linking the antibody to an enzyme. The enzyme, in turn, when exposed to its substrate, will react with the substrate in such a manner as to produce a chemical moiety which can be detected, for example, by spectrophotometric, fluorometric or visual means. Enzymes which can be used to detectably label the detection antibody of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In certain embodiments, the detection antibody is conjugated to alkaline phosphatase, and binding of the detection antibody to the polypeptide-capture antibody complex is detected by adding, for example, disodium 2-chloro-5-(4-methoxyspiro[1,2-dioxetane-3,2′-(5-chlorotricyclo[3.3.1.13.7]decan])-4-yl]-1-phenyl phosphate and measuring chemiluminescence.

Detection may also be accomplished using a radioactively labeled antibody. It is then possible to detect the antibody through the use of radioimmune assays. The radioactive isotope can be detected by such means as the use of a gamma counter or a scintillation counter or by audoradiography. Isotopes which are particularly useful for the purpose of the present invention are 3H, 1311, 35S, 14C, and preferably 1251.

An antibody also can be detectably labeled by coupling it to a chemiluminescent compound. The presence of the chemiluminescent compound-antibody complex is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

### 4. Generating a Standard Curve

Disclosed herein, for example, is a method for generating a standard curve for an Enzyme-Linked Immunosorbent Assay (ELISA) or an AlphaLISA® for detecting a cystic fibrosis transmembrane conductance regulator (CFTR), the method comprising adding a polypeptide disclosed herein to capture antibody (e.g., UNC596); allowing the polypeptide to bind the capture antibody to form a polypeptide-capture antibody complex, adding a detection antibody to the polypeptide-capture-antibody complex; detecting binding of the detection antibody to the polypeptide-capture antibody complex. In some embodiments, the method further comprises the steps of repeating the above steps using varying concentrations of the polypeptide; and generating a standard curve based upon the binding of the polypeptide at the varying concentrations.

In some embodiments, the method further comprises comparing the amount of the binding of the detection antibody to the CFTR protein-capture antibody complex to a standard curve generated as described above.

In certain embodiments, a standard curve is generated using (a) a polypeptide comprising a region exhibiting at least 90% sequence identity to WPSGGQMTGGKRKNSILNPI (SEQ ID NO:1) or to a portion thereof, wherein the portion comprises 15-19 amino acids; (b) a polypeptide exhibiting at least 90% sequence identity to WPSGGQMTGGKRKNSILNPI (SEQ ID NO:1) or to a portion thereof, wherein the portion comprises 15-19 amino acids; or (c) a polypeptide comprising a first region having at least 90% sequence identity to amino acids 1-8 of SEQ ID NO:1 and a second region having at least 90% sequence identity to amino acids 11-20 of SEQ ID NO:1, wherein the polypeptide comprises fewer than 1000 amino acids.

In certain embodiments, the capture antibody binds to amino acids 1-8 of SEQ ID NO: 1, and the detection antibody binds to amino acids 11-20 of SEQ ID NO: 1. In other embodiments, the capture antibody binds to amino acids 11-20 of SEQ ID NO: 1, and the detection antibody binds to amino acids 1-8 of SEQ ID NO: 1.

In some embodiments, the capture antibody is the UNC596 antibody, and the detection antibody is the UNC450 antibody. In some embodiments, the capture antibody is the UNC450 antibody, and the detection antibody is the UNC596 antibody. However, any pair of antibodies recognizing a polypeptide as described herein, can be used in an ELISA or AlphaLISA® assay to detect CFTR or to generate a standard curve as described herein. For example, a pair of antibodies recognizing amino acids 1-8 and 11-20 of SEQ ID NO:1 can be used according to the methods described herein.

In certain embodiments, UNC596 antibody is affixed to a well of a microplate. In some embodiments, the UNC450 antibody is conjugated to alkaline phosphatase. In some embodiments, binding of the UNC450 antibody to the polypeptide-UNC596 complex is detected by adding, for example, disodium 2-chloro-5-(4-methoxyspiro[1,2-dioxetane-3,2′-(5-chlorotricyclo[3.3.1.13.7]decan])-4-yl]-1-phenyl phosphate and measuring chemiluminescence.

In some embodiments, the standard curve is generated by, for example: (a) adding the polypeptide to a container comprising a UNC596 antibody or to a surface to which the UNC596 antibody is affixed; (b) allowing the polypeptide to bind the UNC596 antibody to form a polypeptide-UNC596 complex, (c) adding a UNC450 antibody to the polypeptide-UNC596 complex; (d) detecting binding of the UNC450 antibody to the polypeptide-UNC596 complex.

### 5. Use of a CFTR AlphaLISA® to Identify New CFTR Modulators

The AlphaLISA® is a bead-based assay technology that can be used for screening in a microplate format, e.g., a 384 well plate. The assay is compatible with automation, because it is easy and fast to use, compatible with many types of samples, requires no washing steps, and offers a very high sensitivity and large dynamic range. Thus, the AlphaLISA® can provide a highly sensitive approach for screening new CFTR amplifiers or modulators. Moreover, the AlphaLISA® can be a useful tool for clinical studies.

Screening can be performed according to methods known in the art that are compatible with the AlphaLISA® assay. In one embodiment, a screen is performed by adding cells appropriate for screening to one or more 96 or 384 well plates. Candidate CFTR amplifiers and/or modifiers are added to the plate(s) and the AlphaLISA® assay is performed. Detection of an increase in CFTR expression in the presence of a candidate amplifier and/or modifier as compared to a negative control (e.g., DMSO) is indicative that the candidate molecule may be useful as an amplifier and/or a modifier. Candidate molecules may be further tested for suitability as an amplifier and/or modifier.

Modifiers that increase CFTR expression can be further tested for their ability to increase CFTR activity in primary HBE cells via functional assays such as Ussing chamber electrophysiology. The action of a modifier on CFTR levels can also be explored by measuring whether the modifier modulates CFTR mRNA levels in order to exert its effect on its expression levels. The impact on CFTR protein levels can be investigated using immunoblotting to determine if the modifier influences the maturation of CFTR from a fast migrating species to a slow migrating species in SDS-polyacrylamide gel electrophoresis. Further, modifiers that reduce CFTR protein levels can be investigated for reductions of endogenous CFTR levels in intestinal cell models such as H508, HT-29, and LS411N. The effect of modifiers on CFTR mRNA and immunoblot migration, as discussed above, can also be examined in intestinal cell models.

### 6. Kits

Also provided herein is a kit for performing an ELISA or AlphaLISA® to detect CFTR protein, the kit comprising a polypeptide disclosed herein. In certain embodiments, the kit further comprises a capture and/or detection antibody capable of binding to the polypeptide. In certain embodiments, the capture antibody binds to amino acids 1-8 of SEQ ID NO: 1, and the detection antibody binds to amino acids 11-20 of SEQ ID NO: 1. In other embodiments, the capture antibody binds to amino acids 11-20 of SEQ ID NO: 1, and the detection antibody binds to amino acids 1-8 of SEQ ID NO: 1. In certain embodiments, the capture antibody is a UNC596 antibody and the detection antibody is a UNC450 antibody. In other embodiments, the capture antibody is a UNC450 antibody and the detection antibody is a UNC596 antibody. The kit may further include any of the reagents needed for generating a standard curve and/or performing an ELISA assay to detect and/or quantify CFTR in a sample.

### EXEMPLIFICATION

While this disclosure has been particularly shown and described with references to embodiments thereof, it will be understood by those skilled in the art that various changes in form and details may be made therein without departing from the scope of the disclosure encompassed by the appended claims. The disclosure is illustrated by the following examples which are not meant to be limiting in any way.

### Example 1

### Cell Cultures

A cystic fibrosis bronchial epithelial cell line (CFBE410-) stably expressing either F508del-CFTR (CFBE F508del) or wild type CFTR (CFBE WT) under the control of a human cytomegalovirus (CMV) promoter was used. A CFBE410-cell line that lacks detectable CFTR endogenously was used as a control (CFBE parental).

Primary human bronchial epithelial cells (HBEs) from CF donors homozygous for the F508del mutation (HBE CF), CF donors homozygous for the G542X mutation, and from non-CF donors (HBE WT) were obtained from the CF Center Tissue Procurement and Cell Culture Core of the Cystic fibrosis and Pulmonary Diseases Research and Treatment Center of the University of North Carolina Marsico Lung Institute (Chapel Hill, N.C.). The cells were cultured and differentiated in an air-liquid interface using established procedures (Giuliano et al., SLAS Discov. 2017:2472555217729790).

Primary human airway epithelial cells of nasal origin (HNEs) from CF donors homozygous for the F508del mutation (HNE CF) and non-CF donors (HNE WT) were purchased from Epithelix Sàrl (Geneva, Switzerland). The cells were cultured and differentiated as previously described (Pranke et al. (2017) Sci Rep. 7(1):7375). The primary cells were apically mucous washed for 30 minutes prior to treatment with the amplifier PTI-CH (1, 3, or 10 μM) and DMSO for 24 hours.

### UNC Antibodies

CFTR antibodies UNC154, UNC450, UNC217, and UNC596 were used. The antibodies are available through Cystic Fibrosis Foundation Therapeutics (CFFT) and the University of North Carolina (UNC), see, e.g., www.unc.edu/˜tjjensen/CFADP/antibodies.html.

### CFTR Immunoblotting

Cells were washed with ice-cold phosphate-buffered saline (PBS) and lysed in Pierce™ immunoprecipitation (IP)-Lysis Buffer (Thermo Fisher Scientific Inc., Grand Island, N.Y.) containing protease inhibitor cocktail (cOmplete™ Mini Protease Inhibitor Cocktail tablets, Roche Diagnostics GmBH, Mannheim, Germany). The cells were incubated with lysis buffer for 30 minutes on ice and centrifuged for 30 minutes at 13,000 rpm. Supernatant was collected and protein concentration was assayed by Pierce™ BCA Protein Assay Kit (Thermo Fisher Scientific). The cell lysates were loaded on a NuPAGE 3%-8% Tris-Acetate Gel (Thermo Fisher Scientific), and transferred onto a nitrocellulose membrane (Trans-Blot® Turbo™ Mini Nitrocellulose Transfer Packs, Bio-Rad, Hercules, Calif.). The membrane was blocked in tris-buffered saline with Tween-20 (TBST) and 5% milk and probed with mouse CFTR antibody (UNC596, 1:500 dilution) at 4° C. with gentle shaking overnight. Next day, membrane was washed three times with (TBST). Then, the membrane was incubated for 2 hours with anti-mouse IgG, horseradish peroxidase (HRP)-linked antibody (Cell Signaling Technology, Beverly, Mass.) at 1:2000 dilution. The HRP signal was enhanced by adding SuperSignal™ West Femto Maximum Sensitivity Substrate (Thermo Fisher Scientific) and was visualized using an AlphaInnotech AlphaImager gel imaging system (ProteinSimple, San Jose, Calif.). Actin was used as a loading control.

### Alkaline Phosphatase Labeling of UNC450

UNC450 antibody was diluted to a concentration of 1 mg/mL with PBS. Lightning-Link® Alkaline Phosphatase kit (Innova Biosciences, Cambridge, United Kingdom) was used to conjugate UNC450 with alkaline phosphatase (AP). 1 μL of LL-modifier provided in the kit was added for every 10 μL of antibody and mixed gently. The antibody with LL-modifier was added into the vial containing lyophilized Lightning-Link mix. The contents of the tube were gently mixed and incubated for 3 hours at room temperature. The reaction was terminated by adding LL-quencher (1 μL quencher reagent per 10 μL of antibody). The vial was further incubated for 30 minutes. An equal volume of glycerol was added to obtain a final concentration of 50% (vol/vol). The AP-conjugated UNC450 was aliquoted and stored at −80° C.

### CFTR ELISA

96-well half area microplates (Greiner Bio-One, Monroe, N.C.) were coated with 3 μg/mL of CFTR antibody UNC596 overnight at 4° C. with gentle shaking. Wells were washed 3 times with TBS-T buffer, blocked with SuperBlock™ T20 Blocking Buffer (Thermo Fisher Scientific) and the washing step was repeated. The CFTR standard used was a fusion peptide (SEQ ID NO:1) that is recognized by UNC450 and UNC596 (GenScript, Piscataway, N.J.).

Serial dilutions of CFTR standard, cell lysates, and nasal brushing samples were prepared in IP lysis buffer. 20 μL of lysates, nasal brushing or CFTR standard were added onto the wells and incubated for 2 hours, followed by incubation with the AP-conjugated UNC450 antibody for another 2 hours. The wells were then washed with TBS-T buffer 5 times and detection reagent (CDP-Star™ Substrate with Sapphire-II™ Enhance, Thermo Fisher Scientific) was added for 30 minutes. The assay was read on the EnVision-Multilabel Reader (Perkin Elmer, Waltham, Mass.).

RNA Isolation, Reverse Transcription, and qPCR

For isolation of RNA from cells, the filters were washed with PBS at room temperature. Total RNA was isolated using the Qiagen RNeasy Plus Universal Mini kit (Qiagen, Oslo, Norway). RLT Plus buffer (Qiagen, 250 μL) with 1% 2-Mercaptoethanol (Sigma Aldrich, St. Louis, Mo.) were added onto cells. The cells were scraped and the lysates were transferred into a Qiashredder homogenization column (Qiagen) and spin at 13,000 rpm in a tabletop microfuge for 2 minutes at room temperature. The flow-through was transferred into a collection tube and 1 volume (250 μL) of 70% Ethanol (Sigma Aldrich) was added to the lysate and mixed well by pipetting. The lysate was then transferred onto a RNeasy spin column and spin for 1 minutes at 11,000 rpm. The flow-through was discarded. The column was washed by adding 700 μL of RWT buffer (Qiagen) and was spun at 13,000 rpm for 1 minute and flow-through was discarded. Buffer RPE (500 μL) was added onto column and the sample was centrifuged at 11,000 rpm for 1 minute. The flow-through was discarded. The column was washed one more time with RPE buffer. The RNeasy column was then transferred onto a new collection tube and spin at 13,000 rpm for 2 minutes to dry the membrane. Finally, the RNeasy column was placed into a 1.5 mL collection tube and 30 μL of RNase-free water was added onto the spin column membrane. The membrane was incubated with water for 3 minutes and then centrifuged at 11,000 rpm to elute RNA. The RNA was quantified by NanoDrop (Thermo Fisher Scientific).

For Reverse transcription, 500 ng of total RNA was used for cells and 150 ng of RNA for nasal brushing sample was reverse transcribed using a High-Capacity Transcription Kit (Thermo Fisher Scientific) according to manufacturer's protocol. The CFTR transcript levels were measured using a human-specific CFTR primer (Catalog Number Hs00357011_ml, FAM-labeled Life Technologies). Human Actin B primer (Hs01060665_g1, VIC-labeled, Life Technologies) was used as a reference gene for loading control. The CFTR and Actin primers were mixed with cDNA and Taqman master mix (Applied Biosystems, Foster City, Calif.) was added into a 384-well plate. The samples were run in triplicates on QuantStudio 7 Flex Real-Time PCR System (Applied Biosystems).

### Nasal Epithelial Cell Isolation

Nasal cells were collected from 20 healthy subjects on 4-6 separate occasions over the course of 2 weeks. In brief, the inferior turbinate was visualized and a curette inserted into the nares to brush the turbinate area approximately 5 times. Each nare was brushed with a separate curette and then placed together in an Eppendorf tube containing 350 μL of RLT buffer and 3.5 μL of 2-Mercaptoethanol for subsequent mRNA analyses. The nares were then similarly brushed to obtain additional samples for protein analyses. For protein, each nare was brushed separately with a new curette and then placed together in an Eppendorf tube containing 150 μL of Pierce IP lysis buffer and protease inhibitors. Samples were frozen at −80° C. until processed.

### Detailed CFTR ELISA Protocol

Assay materials and storage conditions are shown in Table 1.

TABLE 1

| Material | Storage | |
|---|---|---|
| Precoated Microplate coated with UNC596 antibody | 2-8° | C. |
| Lyophilized 20X Polypeptide Standard | 2-8° | C. |
| Reconstitute with 1 mL diH₂O | | |
| Wash Buffer 10X Concentrate | 2-22° | C. |
| Pierce IP Lysis Buffer for diluting standards and | 2-8° | C. |
| samples | | |
| 10X Diluent Concentrate for diluting conjugate | 2-8° | C. |
| Alkaline phosphatase Conjugated Detection Antibody | <−80° | C. |
| (UNC450) Concentrate | | |
| CDP-Star substrate | 2-8° | C. |

| | |
|---|---|
| Adhesive plate sealers | ambient |

For reagent preparation, all reagents were brought to room temperature before use. Working dilutions were prepared and used immediately. 20 mL of wash buffer concentrate was diluted with 180 mL of deionized water to prepare 200 mL of wash buffer. 5 mL of 10× HiBlock buffer was diluted with 45 mL of deionized water to prepare 50 mL of 1× HiBlock buffer. The 1× solution was used to prepare the working conjugate solution. For the AP conjugated antibody solution, 12 μL of the conjugate was added to and mixed with 3 mL of the 1× HiBlock buffer.

For the standard curve preparation, lyophilized standard was reconstituted with 1.0 mL of distilled deionized water, or as directed on the label to prepare 10,000 ng/mL stock. The reconstituted standard stock was allowed to sit 5 minutes with occasional gentle agitation prior to making dilutions. Eight standard tubes were labeled as follows: 500, 250, 125, 62.5, 31.3, 15.6, 7.81 and 3.91 ng/mL. 950 μL of IP lysis buffer was pipetted into the 500 ng/mL tube and 500 μL of buffer into each remaining tube. 50 μL of the 10,000 ng/mL stock was transferred to the 500 ng/mL tube and mixed well. 500 μL of the of 500 ng/mL solution was transferred into the 250 ng/mL tube and mixed well. 500 μL of the 250 ng/mL solution was transferred into the 125 ng/mL tube and mixed well. The dilutions were continued in this manner to complete the 2-fold dilution series. The 500 ng/mL solution was set as the highest standard and the IP Lysis buffer was set as the zero standard.

For the assay, all reagents and samples were brought to room temperature before use. All samples, controls, and standards were assayed in duplicate. All reagents, working standards and samples were prepared following the above procedures. 20 μL of standard, control, or sample was added per well. The microplate was covered with an adhesive strip plate sealer and incubated for 3 hours at room temperature on an orbital shaker set to 600+/−50 rpm. 25 μL of the prepared detection antibody solution was added to each well. The microplate was covered with a new adhesive strip plate sealer and incubated for 1 hour at room temperature on an orbital shaker set to 600+/−50 rpm. Each well was aspirated and washed with wash buffer, repeating the process 4 times for a total of 5 washes. Any remaining wash buffer was removed by further aspiration of by inverting the plate and blotting it against clean paper towels. 50 μL of CDP-Star substrate solution was added to each well. The microplate was incubated for 30 minutes at room temperature on the shaker, taking care to protect from light. The chemiluminescence was read within 30 minutes using a microplate reader.

For calculation of the results, the duplicate relative light unit (RLU) readings were averaged for each standard and sample and the average zero standard RLU was subtracted. A standard curve was created by reducing the data using computer software capable of generating a 5PL curve-fit. The standards were fitted to a 5-parameter logistic function with 1/Y2 weighing. The concentrations of the unknown samples were interpolated using the standard curve.

### Results of the CFTR ELISA

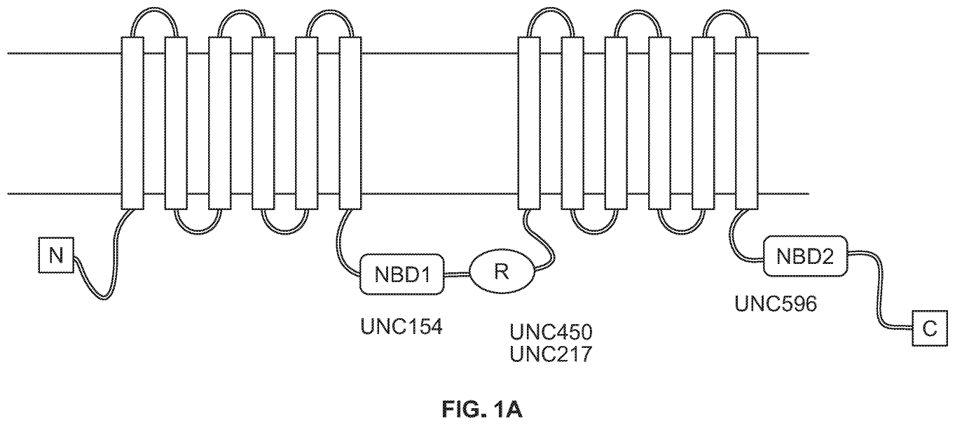

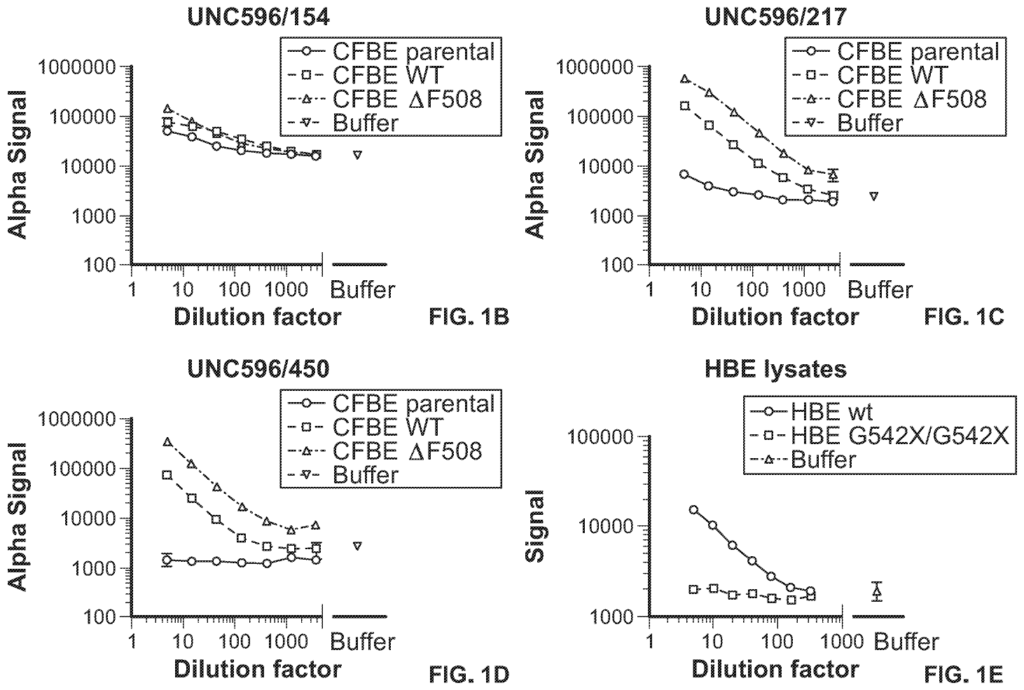

To construct a CFTR-specific protein ELISA, mouse monoclonal antibodies were identified as potential capture and detection reagents (FIG. 1A). For the detection antibody, UNC154, UNC217, and UNC450 were tested. UNC596 as the capture antibody and AP-conjugated UNC450 as the detection antibody provided a large dynamic range of CFBE cells expressing either the wild type or F508del CFTR protein (FIG. 1B). Both cell lines are known to express high levels of the CFTR protein. CFBE parental cells (which do not express CFTR) were used as a control and the values measured were similar to buffer alone. To determine if the CFTR ELISA could detect endogenously expressed CFTR, protein extracts from HBE cultures were analyzed. CFTR protein was detected in protein extracts from HBE WT cell lysate and HBE CF cell lysate, although at a much lower level than CFBE F508del and CFBE WT cells. As a control, HBE cultures containing a homozygous mutation for G542X were analyzed (FIG. 1E). These cells express a truncated CFTR protein which would not be captured by UNC596 (NBD2 domain). F508del CFTR protein could be detected over a 100-fold dilution range over background levels compared to extracts from the HBE-G542X cells. Collectively, the data demonstrated that the prototype CFTR ELISA detects both wild type and F508del CFTR protein from established cell lines and primary bronchial epithelial cells.

As shown in FIG. 1A, CFTR protein consists of five domains: 2 transmembrane domains (TMD), 2 nucleotide-binding domains (NBD1 and NBD2) and a regulatory domain (R domain). Each TMD is composed of 6 transmembrane helices, which form the CFTR channel pore. The 2 TMD domains are connected via NBDs in the cytoplasm. The NBD interacts with nucleotides to regulate the channel activity, which involves opening and closing of the pore. NBD1 is connected to the second TMD via R domain. The R domains regulate the channel activity. The CFTR channel opens only when it is phosphorylated by Protein Kinase A (PKA) and ATP is bound to the NBD domain. The cartoon shows the location where the CFTR antibodies (UNC154, UNC450, UNC217, and UNC596) interact with the CFTR protein. FIG. 1B shows CFTR ELISA results performed on CFBE parental, CFBE WT, and CFBE F508del cells using the UNC596/UNC154 antibody combination. FIG. 1C shows CFTR ELISA results performed on CFBE parental, CFBE WT, and CFBE F508del cells using the UNC596/UNC217 antibody combination. FIG. 1D shows CFTR ELISA results performed on CFBE parental, CFBE WT, and CFBE F508del cells using the UNC596/UNC450 antibody combination. FIG. 1E shows CFTR ELISA results performed on HBE wild type and HBE G542X/G542X cell lysates.

To accurately quantitate CFTR protein expression and control plate-to-plate variation, a 20-amino acid fusion polypeptide (SEQ ID NO:1) was generated which covers the epitopes recognized by UNC450 and UNC596. Using the protocols described above, a standard curve was generated with the fusion polypeptide over a broad concentration range. An overnight incubation of 3 μg/mL coating antibody at 4° C. and 2 hours incubation with lysates, followed by 2 hours with detection antibody at 3 μg/mL produced a reliable assay. The upper (LLOQ) and lower limit of quantitation (LLOQ) were determined to be 1000 ng/mL and 3.7 ng/mL respectively. The % CV was acceptable (<15%) across the linear dilution range of the standard curve.

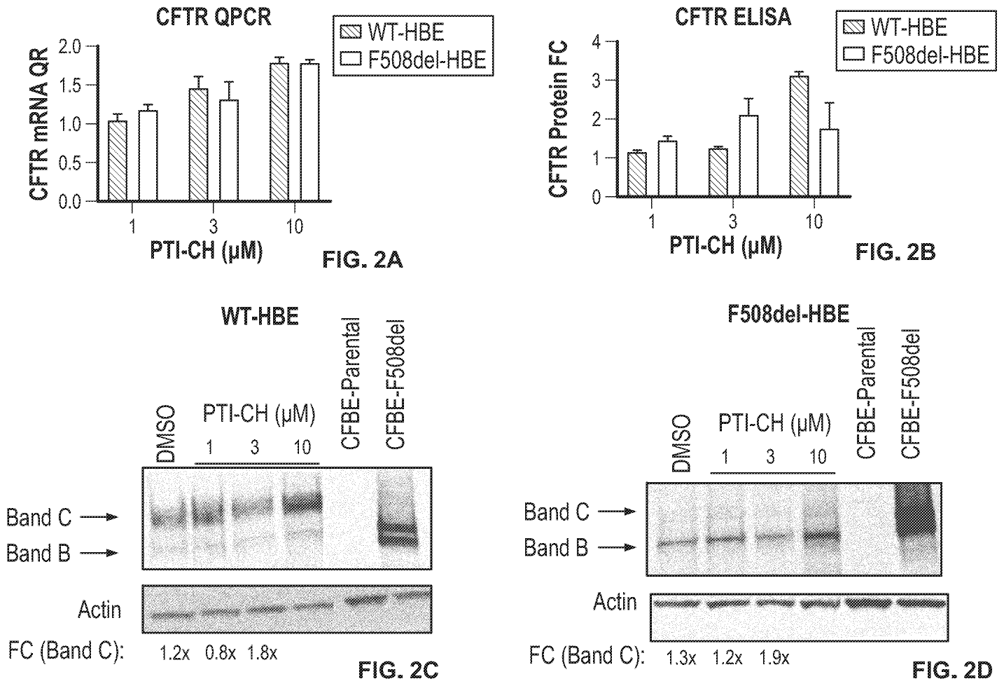

To determine if the ELISA could detect changes in CFTR expression, HBE WT and HBE CF cultures were treated for 24 hours with an amplifier compound. At the end of the 24-hour treatment period, filters were collected for either mRNA or protein analyses. The amplifier increased CFTR mRNA expression in a concentration-dependent manner in both HBE-WT and HBE-CF as detected by qPCR analyses (FIG. 2A). Similarly, protein collected from the filters treated with the amplifier also increased CFTR protein in a concentration-dependent manner as detected by the CFTR ELISA (FIG. 2B). HBE-CF had approximately 25% of F508del protein compared to HBE-WT, indicating that F508del protein is unstable. To confirm these results CFTR protein was also quantitated by western blot analyses using UNC596 antibodies. Quantitation of the CFTR bands B or C was performed by densitometry and normalized to actin protein. HBE-WT or HBE-CF treated with amplifier increased CFTR protein in similar amounts by both western blot and ELISA. (FIG. 2C and FIG. 2D).

WT and F508del human bronchial epithelial cells (HBEs) were treated with 1, 3, and 10 μM of amplifier or DMSO (vehicle) for 24 hours. The total mRNA and protein were collected as described in methods. CFTR/Actin RNA fold changes are shown in FIG. 2A. CFTR ELISA was performed and the CFTR protein levels normalized to total protein concentration assayed by BCA assay, as shown in FIG. 2B. Western blot for CFTR and Actin for WT-HBE and F508del HBEs are shown in FIG. 2C and FIG. 2D, respectively. CFBEs over expressing both WT and F508del CFTR were used as positive controls for CFTR. CFBE parental cells with no CFTR protein expression were used as negative controls for western blots. Fold changes (FC) for CFTR Band B and CFTR Band C are indicated.

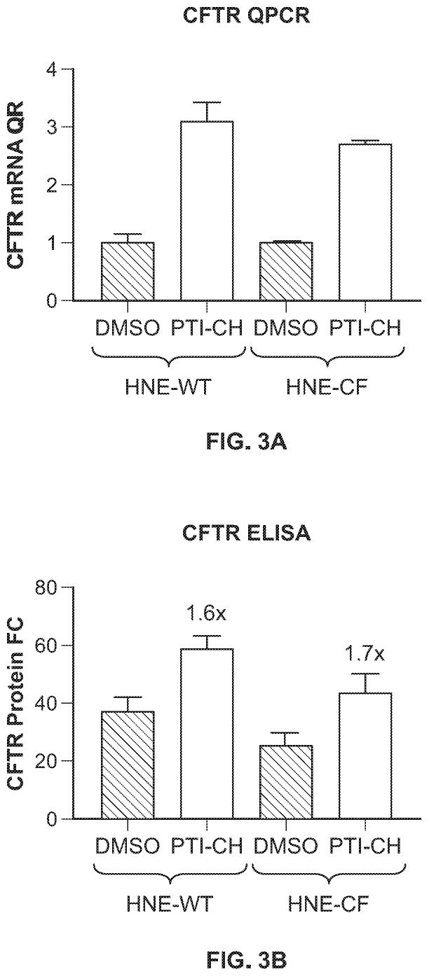

To further confirm this finding, human nasal epithelial (HNE) cultures obtained from a non-CF (HNE-WT) or CF (HNE-F508del) donor were treated with and amplifier compound. Following 24-hour treatment period, filters were analyzed for either mRNA or protein analyses. The amplifier compound increased CFTR mRNA expression approximately 3-fold in both HNE-WT and HNE-CF donors as detected by qPCR analyses, as shown in FIG. 3A, while the CFTR ELISA detected a 2.3-fold and a 1.7-fold increase in CFTR protein in HNE-WT and HNE-CF respectively, as shown in FIG. 3B. To further validate the CFTR ELISA, HNE cell extracts were separated by SDS PAGE and probed with UNC596 antibody. Western blot analyses detected positive control lysates (HBE and CFBE cell lysates), but failed to detect CFTR protein from either HNE WT or HNE CF cells (data not shown). These results further demonstrate that the CFTR ELISA is a sensitive tool to detect changes in CFTR protein expression.

Differentiated nasal cells obtained from healthy subjects (WT-HNEs) and those obtained from homozygous F508del donors (CF-HNEs) were stimulated with amplifier compound (10 μM) or DMSO (vehicle). After 24 hours of treatment, RNA and protein was collected. CFTR qPCR was performed and fold changes in CFTR/Actin RNA were plotted, as shown in FIG. 3A. Protein lysates were extracted for CFTR ELISA. CFTR expression (ng/mL) normalized to total protein content (mg/mL) is represented as CFTR (ng/mg) on the Y-Axis of FIG. 3B.

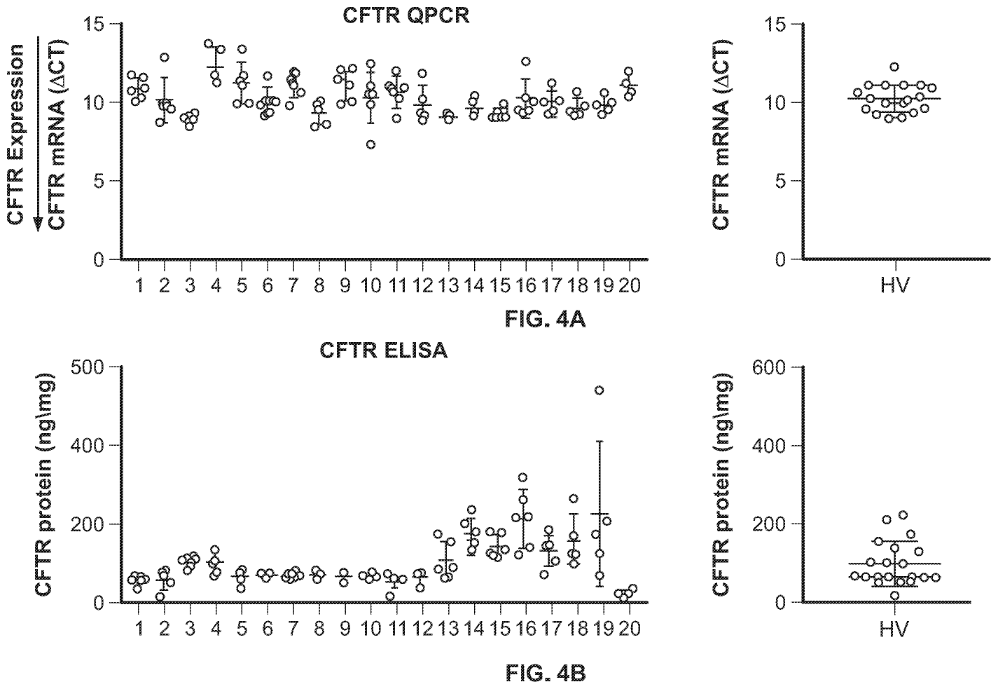

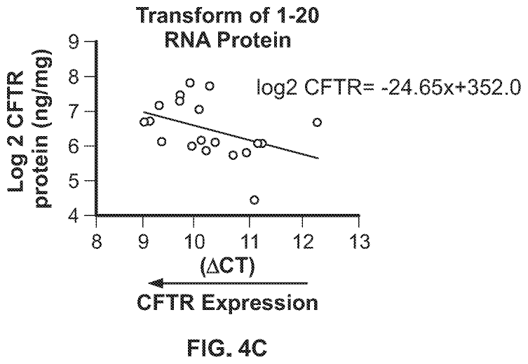

An important application of the CFTR ELISA disclosed herein is to monitor CFTR protein expression and assess the activity of the amplifier in nasal brushing samples. Cells from the inferior turbinate of the nasal cavity are known to express CFTR. To determine if CFTR mRNA and protein can be measured from this region, nasal samples were collected from 20 healthy subjects over a 2-week period. qPCR analyses showed up to a 10-fold difference of inter-subject variability of CFTR mRNA levels between healthy subjects, as shown in FIG. 4A, and the % CV for intra-subject variability was 8%. All nasal epithelial samples collected from healthy subjects had detectable CFTR protein levels as measured by CFTR ELISA. The amount of CFTR protein quantitated by CFTR ELISA also showed a wide range of CFTR expression ranging from 20 to 220 ng/mg but low intra-patient variability, as shown in FIG. 4B. To determine if CFTR mRNA and protein expression were regulated similarly, relative CFTR mRNA and protein expression were compared across all healthy subjects. There was a weak positive correlation between CFTR mRNA and protein, i.e., higher CFTR mRNA levels (low DCT) correlate with higher CFTR protein, as shown in FIG. 4C. The data set suggests that CFTR mRNA and protein expression can be monitored and quantitated by qPCR and ELISA respectively over a broad range in nasal samples from healthy subjects.

Nasal cells were collected from 20 healthy volunteers (HV) for mRNA or protein on 4-6 on separate occasions over 2-week period. CFTR expression was measured by qPCR using actin as a house keeping gene, as shown in FIG. 4A. The CFTR protein expression was analyzed by CFTR ELISA and normalized to protein content, as shown in FIG. 4B. Correlation between CFTR protein (Y-Axis) and CFTR mRNA (X-Axis) is shown in FIG. 4C.

### Example 2

This example shows that CFTR can be quantified across multiple cell lines using an AlphaLISA® in accordance with the disclosure herein.

In this assay, streptavidin-coated donor beads are coated with a biotinylated antibody that recognizes the CFTR protein and acceptor beads are coated with a second antibody that recognizes the CFTR protein. The AlphaLISA® assay uses luminescent oxygen-channeling chemistry in which laser irradiation of donor beads at 680 nm causes chemiluminescent emission from the acceptor bead at 615 nm. Thus, when CFTR protein is present, both donor and acceptor beads bind the analyte. Their close proximity triggers a reaction that results in chemiluminescent emission at 615 nm from the acceptor bead.

### Materials and Methods

The following reagents are used in the AlphaLISA® assay: IP lysis buffer (Life ˜87788), CFTR Recombinant fusion protein (Genscript, Piscataway, N.J.); Opti-plates 384 (Perkin Elmer, Waltham, Mass.; 6007299); conjugated acceptor beads with UNC596 antibody (5 mg/ml); biotinylated UNC450 antibody (0.08 mg/ml); 10× AlphaLISA® immunoassay buffer (Perkin Elmer; AL000F); cOmplete™, ULTRA, Mini, EDTA-free, EASYpack Protease Inhibitor Cocktail (Roche Diagnostics GmBH, Mannheim, Germany; 05892791001); and Streptavidin Donor Beads (Perkin Elmer; 6760002B).

CFBE F508del cells were treated with either PTI-CH (amplifier) or vehicle as described in Example 1. Cells were washed with 2× cold PBS then lysed with IP buffer containing protease inhibitors. Plates were removed from incubator and placed on ice. Using a multichannel pipette, all media was removed from the wells and cells were rinsed 2× with 100 μL/well cold PBS. All PBS was removed and 100 μL/well Lysis Solution was added using a multichannel pipette.

The bottom of the wells was scraped using the pipette tips and then the contents of the well were pipetted up and down about 10 times. Lysates were left in the tissue culture plate, sealed with a plastic sealing film and stored at −80° C.

A serially diluted CFTR fusion peptide (as described herein as SEQ ID NO:1) was used as standard for assay. A 1000 ng/ml stock of CFTR fusion peptide was made from the original peptide concentration. Standard dilutions were made in 1× AlphaLISA® immunoassay buffer. Serial dilutions of 1:2 were made down 10 standard points as shown in Table 2. The 11th standard is designated as “Blank” (only contains 1× dilution buffer).

TABLE 2

| | Concentration |
|---|---|
| STD# | (ng/ml) |
| 1 | 1000 |
| 2 | 500 |
| 3 | 250 |
| 4 | 125 |
| 5 | 62.5 |
| 6 | 31.25 |
| 7 | 15.625 |
| 8 | 7.8125 |
| 9 | 3.90625 |
| 10 | 1.95313 |
| 11 | Buffer only (blank) |

Samples are diluted as shown in Table 2 using AlphaLISA® assay buffer in a 96 well polypropylene plate and mixed by pipetting up and down 10 times. The dilutions are empirically tested according to each cell line being used.

An acceptor bead master mix was prepared. For one 384 well plate, 5900 μL of AlphaLISA® assay buffer was mixed with 30 μL biotinylated antibody and 30 μL acceptor beads. The tube was inverted 10 times to mix.

Twenty μL of master mix was transferred into each well of a clean Opti-plate 384. Five μL each of standards, samples, and blanks (buffer only) were transferred to the master mix assay plate. The plate was covered with plastic sealer and mixed on a plate shaker for 1 min. Plate was incubated at room temperature either 2 h or overnight without shaking.

In a dark room, donor beads were prepared by mixing 7100 μL of AlphaLISA® assay buffer and 113 μL streptavidin donor beads and inverting the tube 10 times. The plastic plate sealer was removed and 25 μL of donor beads added to each well. The plate was covered with plastic sealer and wrapped with aluminum foil and mixed on a plate shaker for 30 min.

### Results

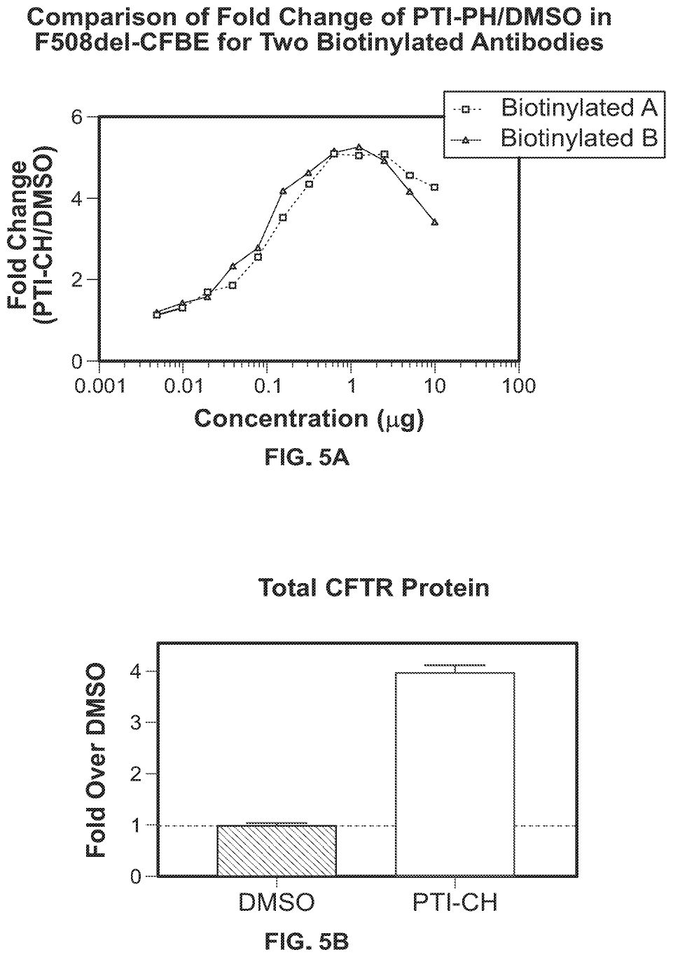

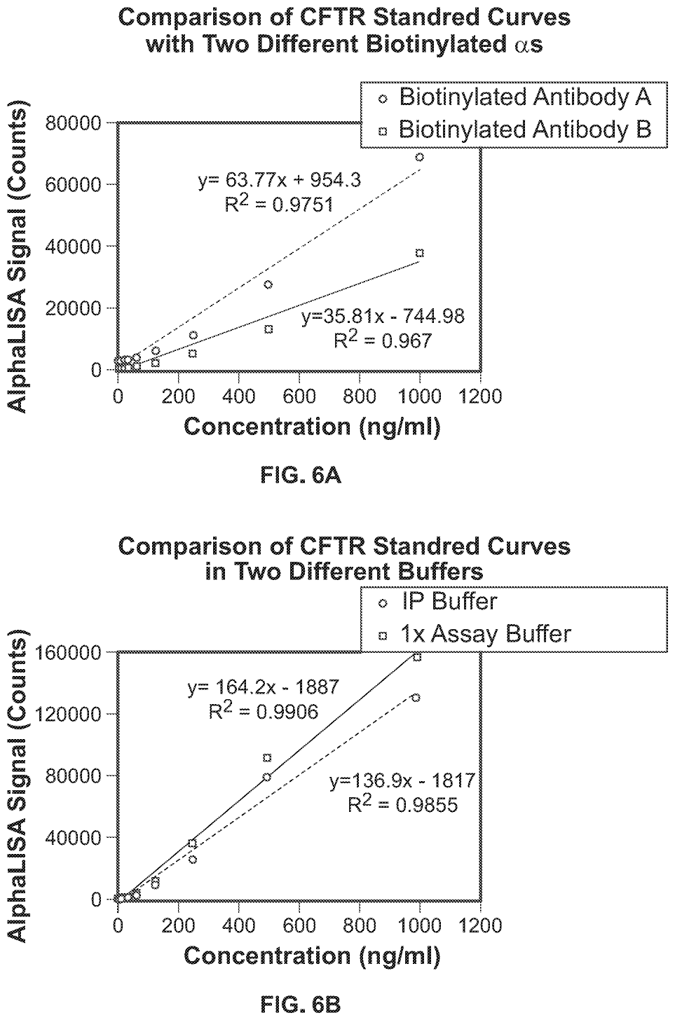

The plate was read on a Perkin Elmer EnVision (or a similar instrument can be used) at 615 nm (AlphaLISA® Opti-384 program). As shown in FIGS. 5A and 5B, the results were comparable to those from the ELISA assay presented in Example 1, and the results were equivalent regardless of whether UNC450 was biotinylated or UNC596 was biotinylated. Further evidence showing that either antibody could effectively function as the biotinylated antibody is shown in FIGS. 6A and 6B. As shown in FIG. 6A, standard curves were similar whether UNC596 was the biotinylated (donor) antibody (circles) or UNC450 was the biotinylated (donor) antibody (squares).

Further, standard curves were similar regardless of assay buffer used. Specifically, as shown in FIG. 6B, generation of a standard curve was similar whether Pierce IP Lysis buffer or 1× AlphaLISA® buffer was used.

### Example 3

To determine whether an AlphaLISA® assay could be used to screen for and identify new amplifiers, the assay described in Examples 1 and 2 was performed in three intestinal carcinoma cell lines, H508, HT-29 and LS411N, as well as in F508del-CFBE and F508del-HBE. Results from the AlphaLISA® assay are shown in Table 3.

TABLE 3

| Cell Line: | Fold change (PTI-CH/DMSO) | ns |
|---|---|---|
| H508 | 1.3 ± 0.08 | 9 |
| HT-29 | 1.7 ± 0.05 | 9 |
| LS411N | 1.9 ± 0.13 | 7 |
| F508del-CFBE | 4.9 ± 0.53 | 3 |
| F508del-HBE | 1.4 ± 0.07 | 3 |

Western blot analysis was performed as a control and showed similar fold changes in CFTR expression in response to the PTI-CH amplifier; specifically, 1.4× for H508, 1.4× for HT-29, and 2.2× for LS411N. Accordingly, these results demonstrate that the AlphaLISA® assay can be used effectively for screening for CFTR amplifier molecules using a variety of cell lines that express CFTR. These results also demonstrate that the AlphaLISA® assay is capable of detection of endogenous CFTR and overexpressed CFTR, and that it detects CFTR in cells derived from lung and non-lung tissues, such as intestine. Accordingly, in certain embodiments screening can be used to detect modifiers that reduce CFTR levels in the digestive system. Such modifiers may be desirable for indications such as cholera-induced diarrhea.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A polypeptide comprising a first region having at least 90% sequence identity to amino acids 1-8 of SEQ ID NO:1 and a second region having at least 90% sequence identity to amino acids 11-20 of SEQ ID NO:1, wherein the polypeptide comprises fewer than 1000 amino acids.

2. A polypeptide comprising a region exhibiting at least 90% sequence identity to WPSGGQMTGGKRKNSILNPI (SEQ ID NO:1) or to a portion thereof, wherein the portion comprises 15-19 amino acids.

3. A polypeptide exhibiting at least 90% sequence identity to WPSGGQMTGGKRKNSILNPI (SEQ ID NO:1) or to a portion thereof, wherein the portion comprises 15-19 amino acids.

4. The polypeptide of claim 1, wherein the at least 90% sequence identity is at least 95% sequence identity.

5. The polypeptide of claim 1, wherein the at least 90% sequence identity is at least 98% sequence identity.

6. The polypeptide of claim 1, wherein the polypeptide is capable of binding to a UNC596 antibody and a UNC450 antibody.

7. A nucleotide encoding the polypeptide of claim 1.

8. A vector comprising the nucleotide sequence of claim 7.

9. A cell expressing the vector of claim 8.

10. A method for generating a standard curve for an Enzyme-Linked Immunosorbent Assay (ELISA) or an AlphaLISA® for detecting a cystic fibrosis transmembrane conductance regulator (CFTR), the method comprising:
(a) adding the polypeptide of claim 1 to a container comprising a capture antibody;
(b) allowing the polypeptide to bind the capture antibody to form a polypeptide-capture antibody complex,
(c) adding a detection antibody to the polypeptide-capture antibody complex; and
(d) detecting binding of the detection antibody to the polypeptide-capture antibody complex.

11. The method of claim 10, further comprising the steps of:
(e) repeating steps (a) through (d) using varying concentrations of the polypeptide; and
(f) generating a standard curve based upon the binding of the polypeptide at the varying concentrations.

12. The method of claim 10, wherein the capture antibody is UNC596 and/or the detection antibody is UNC450.

13. The method of claim 12, wherein the UNC596 antibody is affixed to a well of a microplate.

14. The method of claim 12, wherein the UNC450 antibody is conjugated to alkaline phosphatase.

15. A method for quantifying CFTR protein expression in a sample, the method comprising the steps of:
(a) adding a sample containing the CFTR protein to a capture antibody, wherein the capture antibody is UNC596;
(b) allowing the CFTR protein to bind the UNC596 antibody to form a CFTR protein-UNC596 complex,
(c) adding a detection antibody to the CFTR protein-UNC596 complex, wherein the detection antibody is UNC450; and
(d) detecting binding of the UNC450 antibody to the CFTR protein-UNC596 complex.

16. The method of claim 15, further comprising comparing the amount of the binding of the UNC450 antibody to the CFTR protein-UNC596 complex to a standard curve generated using:
(a) a polypeptide comprising a region exhibiting at least 90% sequence identity to WPSGGQMTGGKRKNSILNPI (SEQ ID NO:1) or to a portion thereof, wherein the portion comprises 15-19 amino acids;
(b) a polypeptide exhibiting at least 90% sequence identity to WPSGGQMTGGKRKNSILNPI (SEQ ID NO:1) or to a portion thereof, wherein the portion comprises 15-19 amino acids; or
(c) a polypeptide comprising a first region having at least 90% sequence identity to amino acids 1-8 of SEQ ID NO:1 and a second region having at least 90% sequence identity to amino acids 11-20 of SEQ ID NO:1, wherein the polypeptide comprises fewer than 1000 amino acids.

17. The method of claim 16, wherein the standard curve is generated by:
(a) adding the polypeptide to a container comprising a second UNC596 antibody or to a surface to which the second UNC596 antibody is affixed;
(b) allowing the polypeptide to bind the second UNC596 antibody to form a polypeptide-UNC596 complex,
(c) adding a second UNC450 antibody to the polypeptide-UNC596 complex; and
(d) detecting binding of the second UNC450 antibody to the polypeptide-UNC596 complex.

18. The method of claim 17, wherein the second UNC596 antibody is affixed to a well of a microplate and/or wherein the second UNC450 antibody is conjugated to alkaline phosphatase.

19. A kit for performing an ELISA or an AlphaLISA® to detect CFTR protein, the kit comprising:
(a) a polypeptide of claim 1;
(b) a capture antibody; and
(c) a detection antibody.

20. The kit of claim 19, wherein the capture antibody is UNC596 and the detection antibody is UNC450.
